(19) United States
(12) Patent Application Publication
(10) Pub. No.: US 2026/0273249 A1
(43) Pub. Date: Sep. 17, 2026

(54) SYSTEMS AND METHODS FOR INTRAPARENCHYMAL DELIVERY OF THERAPEUTIC OR DIAGNOSTIC AGENTS

(71) Applicant: BIONAUT LABS LTD., Herziya (IL)

(72) Inventors: Alex KISELYOV, San Diego, CA (US); Florent CROS, Los Angeles, CA (US); Michael KARDOSH, Kiryat Ono (IL); Michael SHPIGELMACHER, Los Angeles, CA (US); Jason SHIH, Pasadena, CA (US); Darrell HARRINGTON, Canoga Park, CA (US); Suehyun CHO, Torrance, CA (US); Roberta POCEVICUITE, Pasadena, CA (US); William G. LOUDON, San Clemente, CA (US)

(73) Assignee: BIONAUT LABS LTD., Herzliya (IL)

(21) Appl. No.: 19/469,287

(22) PCT Filed: Mar. 26, 2024
(86) PCT No.: PCT/US2024/021538

§ 371 Date: Sep. 25, 2025

Related U.S. Application Data
(60) Provisional application No. 63/456,330, filed on Mar. 31, 2023.
(60) Provisional application No. 63/621,913, filed on Jan. 17, 2024.
(60) Provisional application No. 63/562,218, filed on Mar. 6, 2024.

(51) Int. Cl. A61M 31/00; A61B 17/34; A61B 34/00; A61B 34/10; A61B 34/20; A61B 90/00

(52) U.S. Cl. CPC A61M 31/002; A61B 34/20; A61B 34/73; A61B 17/3468; A61B 2034/107; A61B 2090/376; A61M 2210/0693

(57) ABSTRACT

Provided are systems and untethered miniature devices for delivering therapeutic and/or diagnostic agent(s) and/or activating agent(s) that converts a prodrug into the therapeutic and/or the diagnostic agent(s) to a target volume of tissue, such as brain tissue, in a patient. The miniature devices may be placed in or near the targeted volume of the tissue and are configured to form a channel along a curvilinear path within that volume. The channel is then filled with desired amount of the agent(s), thereby allowing them to diffuse into the targeted volume to follow a specific treatment regimen.

### FIELD OF THE INVENTION

The presently disclosed subject matter relates to systems configured to form an intraparenchymal channel within a target volume of solid tissue, such as the brain, in a patient that is then filled with a medical material. For example, such systems usmagnetic fields to direct operation of an untethered miniature devices within the patient.

### BACKGROUND OF THE INVENTION

The dispersion of a therapeutic agent within a mass of tissue (e.g., the brain, liver, etc.), once the agent has been injected at a single given site (or locus) is governed by myriads of factors and mechanisms including laws of diffusion, mass transport, interstitial fluid flow, other local physiological and biological processes that are not controlled.

Specifically, if left “un-aided” (e.g., in the absence of a continuous pressure from a syringe), a finite bolus of a therapeutic agent (liquid or solid that dissolves) might not distribute beyond the location of the initial locus as dictated by biophysical (e.g., Fick's law of diffusion) and physiological conditions. This is especially true when the liquid contains large biologic molecules (MW>>1,000 D) and cells. By way of example, a crude estimation for a biologic entity as large as an adenovirus based purely on Ficks' law of diffusion leads to rates of 0.2-0.5 mm/day. It is generally accepted that the larger the size/molecular weight or related biophysical parameters of a therapeutic entity, the slower it will move through any given tissue. In this case, the term “move” refers to the ability for the virus to migrate some distance away from the site of initial injection, after a specific or arbitrary amount of time.

The more limited the ability to “move,” the more limited the volume of tissue a particular virus can be expected to affect. When the virus is used to deliver a particular biochemical molecule of therapeutic value to a group of cells, the ability to move is therefore potentially limiting the ability of the virus to therapeutically “impact” tissue volume. By way of example, the average size of diagnosed and treated brain tumor such as a glioblastoma multiform (GBM) is between single unit to few tens of cubic centimeters.

### SUMMARY OF THE INVENTION

According to an aspect of the presently disclosed subject matter, there is provided a method for delivering a medical material to a target volume of solid tissue in a patient, the method comprising:
forming an intraparenchymal channel along a predefined path, at least a portion of the predefined path being within or adjacent to the target volume; and
delivering the medical material via the channel to the target volume.

The delivery of medical material may comprise administering a fluid or gel comprising a therapeutic and/or diagnostic agent into the channel.

The therapeutic and/or diagnostic agent may be a small molecule, a peptide, a peptoid, an oligonucleotide sequence, a nucleic acid, an oncolytic virus, an endogenous cell, and/or an engineered cell.

The delivery of medical material may comprise administering a fluid or gel comprising an activating agent that converts a prodrug into a therapeutic and/or diagnostic agent into the channel. The administering may comprise pumping the fluid or gel into the channel.

The delivery of medical material may comprise inserting one or more surgical tools through the channel to perform therapeutic delivery or microsurgery at a target location within or adjacent to the target volume.

The delivery of medical material may comprise installing one or more neurostimulation and/or neurodiagnostic devices in the channel at the target location.

The delivery of medical material may comprise navigating an untethered treatment miniature device to the target location via the channel.

The method may further comprise:
providing a system comprising:
   a miniature device configured to be maneuvered by magnetic fields; and
   an external system configured to generate the magnetic fields to propel and navigate the miniature device in the target volume of tissue; and
operating the external system to propel and navigate the miniature device to travel along the predefined path to form the channel.

The same or an additional miniature device may control local exposure to the therapeutic agent to spare healthy tissue.

The method may further comprise retrieving the miniature device from the patient before delivery of medical material.

Retrieving the miniature device may include navigating and propelling the miniature device into a needle.

The miniature device may have a corkscrew shape or a wood screw shape.

The method may further comprise imaging the miniature device in real time within the target volume.

The method may further comprise:
receiving output from a biplane X-ray system communicatively coupled to a control system of a magnetic propulsion system that propels the miniature device; and
determining, within the control system from the output, a position and orientation of the miniature device within the patient.

The determining may comprise determining a relative location of an array of magnetic coils in reference to the miniature device from the output from the biplane X-ray system; and adjusting propulsion parameters based on the relative location.

The system may further comprise a needle having a proximal end and a distal end and a lumen extending from the distal end to the proximal end, the method further comprising:
introducing the needle through a burr hole in the patient's skull, such that the proximal end resides outside the patient's body and the distal end is placed near or within the targeted volume brain tissue; and
inserting the miniature device into the proximal end of the needle and releasing the miniature device from the distal end of the needle into a starting location of the predetermined path.

The needle may be introduced via a trans-peduncular approach.

The solid volume may be in brain tissue.

The target volume may comprise tumor tissue. The tumor tissue may be a brainstem glioma. The brainstem glioma may be diffuse intrinsic pontine glioma.

The tumor tissue may be in the pons of the patient.

The predetermined path may comprise a curvilinear portion.

The curvilinear portion of the predetermined path may be within the target volume.

The curvilinear portion may be three-dimensional.

The method may further comprise the step of administering the prodrug to the patient.

The activating agent may comprise an enzyme and/or an enzymatically active oligonucleotide.

The activating agent may encode an auxiliary activating agent, the auxiliary activating agent converting the prodrug into the therapeutic agent by directly interacting with the prodrug.

An extracellular, intracellular, and/or intranuclear process may express the auxiliary activating agent encoded by the activating agent.

The activating agent may comprise an enzyme precursor, an oligonucleotide precursor, and/or a protease precursor.

The activating agent may further comprise a vector configured for cellular delivery of the activating agent.

The vector may be selected from a group including an adeno-associated virus, a human immunodeficiency virus, a human papillomavirus sequence, one or more small molecules, one or more lipids, a peptide sequence, and a recognition sequence.

The activating agent may be and/or encode one or more enzymes selected from a kinase, a phosphatase, a peptidasc, a ligasc, a lyasc, a hydrolasc, a protcasc, a deacctylasc, a phosphodiesterase, an esterase, an amidase, a reductase, a phospholipase, or a cytochrome.

The prodrug may be administered before the activating agent.

The prodrug may be administered systematically.

According to another aspect of the presently disclosed subject matter, there is provided a system configured for performing the method according to any one of the preceding claims.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present disclosure.

FIGS. 1A-1B depict a non-limiting example of a treatment using a miniature device (indicated as “bot” in the figures for brevity) of the present disclosure. FIG. 1A depicts the untethered miniature device, delivered in the tumor using a biopsy-like tool, traveling in a tumor, and creating a channel. Once the channel is completed, it is irrigated and filled with a therapeutic agent, using the same biopsy-like tool. FIG. 1B depicts that in the hours post irrigation, the therapeutic liquid continues to diffuse deeper in the tumor.

FIGS. 2A-2D depict non-limiting examples of three miniature devices of the present disclosure. FIG. 2A depicts a miniature device that is shaped like a corkscrew. FIG. 2B is a cross section through the corkscrew shaped miniature device in the presence of a rotating magnetic field (B). FIG. 2C depicts a miniature device that resembles a wood screw. FIG. 2D depicts a miniature device that resembles a bullet.

FIGS. 3A-3G illustrate a non-limiting example of a method of using a system of the present disclosure. FIG. 3A depicts a tumor in the pons of a subject. FIG. 3B illustrates a needle accessing the tumor through a burr hole. FIG. 3C illustrates a miniature device being introduced through the needle near or inside the tumor. FIG. 3D illustrates the miniature device travelling due to an external force along a curvilinear path and generating a channel inside or near the tumor.

FIG. 3E depicts removal of the miniature device from the tissue. FIG. 3F depicts the filling of the channel created by the miniature device with a liquid therapeutic agent introduced through the needle. FIG. 3G depicts removal of the needle and diffusion of the agent into the tissue.

FIG. 4 schematically illustrates an external system for providing a treatment to a target volume in accordance with embodiments of the present disclosure.

FIG. 5 schematically illustrates an external system for providing a treatment to a target volume in accordance with embodiments of the present disclosure.

FIG. 6 is a block diagram illustrating an external system for providing a treatment in accordance with embodiments of the present disclosure.

FIG. 7 illustrates a block diagram of an example machine upon which any one or more of the procedures or methods discussed herein may be performed in accordance with embodiments of the present disclosure.

FIGS. 8A through 8E illustrate further examples of methods of using the system according to the present disclosure.

FIGS. 9 through 10E illustrate methods of using the system, including use of an introduction tool.

### DETAILED DESCRIPTION OF THE INVENTION

As used here, including the appended claims, the singular forms of words such as “a,” “an,” and “the,” include their corresponding plural references unless context clearly dictates otherwise.

All references cited herein are incorporated by reference to the same extent as if each individual publication, patent application, or patent, was specifically and individually indicated to be incorporated by reference.

In one aspect, provided herein are devices and systems that comprise a millimeter-scale or sub-millimeter-scale tetherless object controlled or manipulated remotely by an external force(s) including but not limited to magnetic, radiofrequency, piezoelectric or alternative force field, referred to herein as the “miniature device,” and separate from the miniature device an interactive hardware-software platform, referred to herein as the “external system,” that generates, modulates, and controls magnetic or alternative driving force fields in a defined three-dimensional operational volume to propel and navigate the miniature device to a specific anatomical target to complete a mission. In particular, a system may be provided according to the presently disclosed subject matter, comprising a suitable external system and one or more suitable miniature devices. An example of such a system is described in PCT application PCT/US2022/40303, the full contents of which are incorporated herein by reference in its entirety.

The miniature device is propelled and navigated remotely by the external system through specific anatomical milieu, including, but not limited to, the lumen, cavity, vessel, tissue(s), and circuitry (e.g., a network of neurons within brain tissue of a patient). The milieu can include homogeneous or heterogeneous components, such as lumens, respective luminal lining, and adjacent tissue(s). One such heterogencous compartment is the central nervous system (CNS).

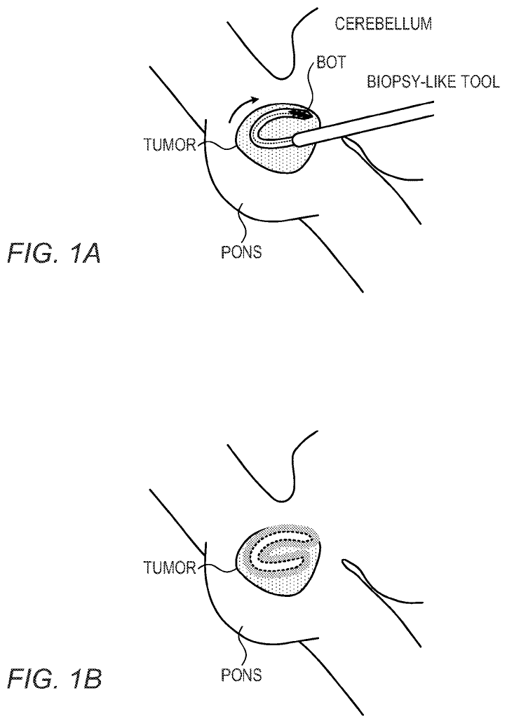

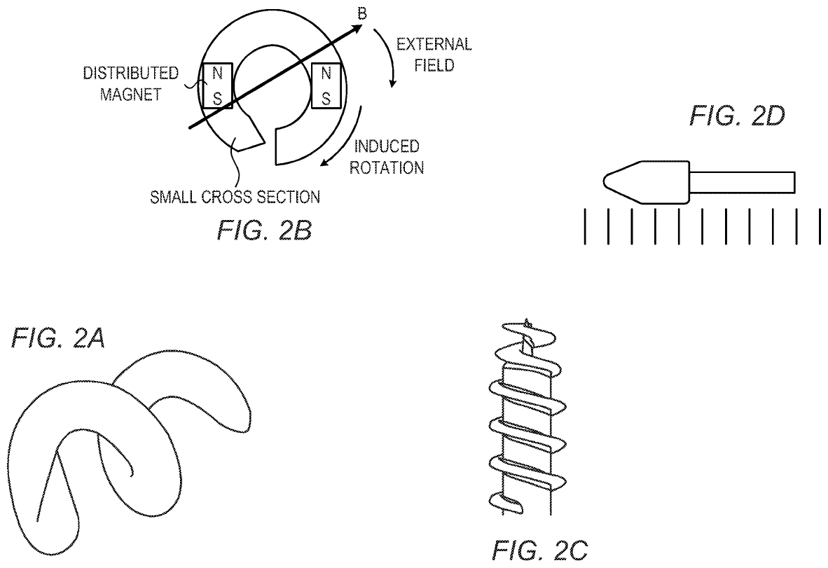

The miniature device follows a predicted path in tissue, e.g., brain tissue; the path could be linear or curvilinear as shown on FIGS. 1 and 2.

The miniature device is introduced through a needle or canula at or near a target volume and then navigated through a healthy and/or pathological milieu to create a channel in the target volume. A therapeutic and/or diagnostic agent and/or an activating agent that converts the prodrug into the therapeutic and/or the diagnostic agent may be subsequently administered to fill the channel and then diffuse into diseased tissue at or near the target volume. According to some examples, the pathological or diseased tissue is a malignant glioma (MG), for example comprising a malignancy. The MG may be a diffuse intrinsic pontine glioma, glioblastoma, or a focal glioma, for example located in the cortex or pons part of the brainstem. The therapeutic or diagnostic agent may comprise one or more chemical compounds, one or more small molecules, biologics, cells, one or more radioisotopes, combination thereof, etc., for treating the diseased tissue.

Embodiments of the present disclosure include the use of tools, methods, and routes commonly practiced and known as safe to reach selected or sought after regions of the brain.

The tools of the present disclosure include biopsy needles or other needles that have similar profiles and attributes. The tool may be designed with specific features to minimize tissue damage, along the path to the region of interest. The tool may have a lumen that extends from its proximal end to its distal end. This lumen allows fluid or devices, such as the miniature devices described herein, to travel from one end of the tool that resides outside the body to the other end.

Routes include, but are not limited to, keyhole access points (i.e., a procedure in which brain and/or skull base tumors are removed through smaller, more precise openings that minimize collateral damage to the surrounding scalp, brain, blood vessels, nerves, etc.), trans-peduncular trajectories used for accessing and biopsying the pons in a human, and/or any other suitable routes.

Embodiments of the present disclosure allow the delivery of a tetherless miniature device directly to the region of interest. The miniature device is designed so that it can be coaxed into travelling in the tissue. As the tetherless miniature device moves within the tissue, the tissue is displaced, and as a result a channel of a desired profile length and topology is created within the tissue itself, i.e., the miniature device forms the channel by boring through solid (non-fluid) tissue. The channel created by the miniature device exhibits a linear or curvilinear path, which is filled with a therapeutic, a diagnostic or an activating agent that can be delivered via conventional devices. The topology of the channel may be controlled or designed to maximize therapeutic or diagnostic effects. The channel may maintain the desired topology or self-repair (close) for desired interval of time (seconds, hours, days, wccks, months, ycars).

It will be appreciated that herein the specification and appended claims, the term “curvilinear” includes paths which are curvilinear, as well as paths which include linear and curvilinear segments. Moreover, curvilinear paths described herein may be two- or three-dimensional, unless otherwise clear from context.

In some embodiments of the present disclosure, the miniature device is coaxed to return to the distal end of the tool. In some embodiments, the miniature device travels back to the tool along the channel.

In some embodiments of the present disclosure, a liquid agent is injected through the lumen of the tool and forced into the channel. In some embodiments the agent is a therapeutic agent. In some embodiments, the agent is a diagnostic agent. The pressure, flow, concentration, nature, viscosity, etc., of the fluid can be chosen to meet the needs of the user.

The fluid is allowed time to fill the channel. Diffusion of some chemical or non-chemical substances dissolved or mixed with the liquid is allowed to occur naturally.

In some embodiments of the present disclosure, a formulated agent is injected through the lumen of the tool and forced into the channel. The formulation may include, but is not limited to, controlled release and/or slow-release gels. The pressure, flow, concentration, nature, viscosity, therapeutics release kinetics, etc., of the formulation can be chosen to meet the needs of the user.

The foregoing approach of the present disclosure has the advantage of allowing a medical practitioner to bring the miniature device directly to the region of interest along a complex curvilinear path using standard techniques and known access routes inside tissue, such as the brain. Thus, the risk of damage along the way to the region of interest is known and well understood. In addition, the channel formed by the miniature device can be used to reach locations of interest inside the tissue which would otherwise be difficult to access with conventional needle- or catheter-based approaches.

The channel can be refilled with therapeutics and/or diagnostic agents and/or activating agents that convert a prodrug into the therapeutic and/or the diagnostic agent as needed to maintain the required treatment regimen.

If necessary, the channel may be repaired by navigating a miniature device along the original path of the channel. In addition, the channel may be extended, e.g., by navigating a miniature device within the tissue beyond the original path, and/or expanded, e.g., by navigating a larger miniature device along the original path.

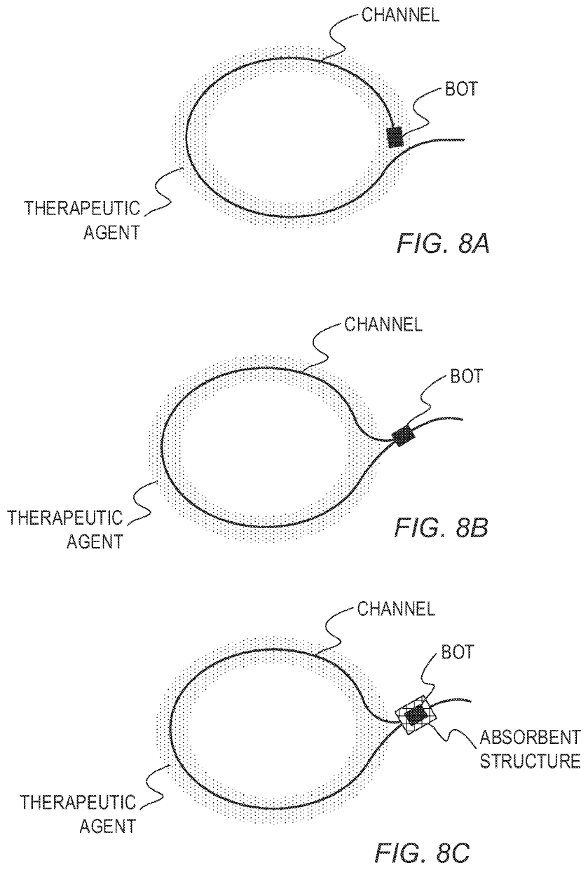

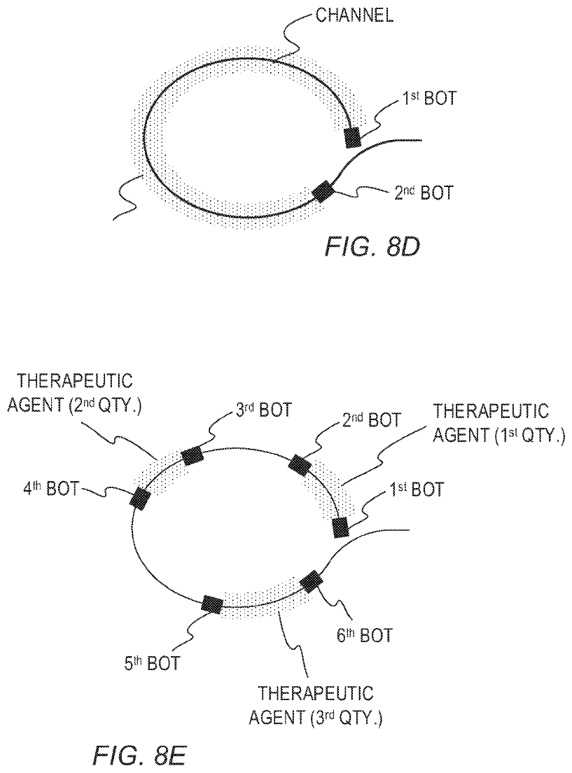

Local concentration of therapeutics or diagnostic in the channel could be controlled by the same or an additional miniature device as shown in FIGS. 8A through 8E. Specifically, the miniature device may act as a plunger, blotter, stopper or scrubber as shown FIGS. 8A through 8E. This is advantageous to control local exposure of healthy tissue adjacent to the channel. This is particularly advantageous for the treatment of diffuse conditions in the tissue that exhibit specific loci of disease.

The curvilinear channels made by the miniature device could also be used to introduce tethered devices as well; these tethered devices may include commercial neurovascular catheters (Integer, www.integer.net/products/neurovascular-microcatheters/default.aspx; Stryker, www.stryker.com/us/en/neurovascular.html) to perform local microsurgery or local delivery of therapeutic/diagnostic agent(s)

The curvilinear channels made by the miniature device could also be used for placement of neurostimulation or neurotransmitting devices, which may be placed temporarily or permanently, as needed.

curvilinear channels made by the miniature device could also be used to insert one or more surgical tools through the channel to perform therapeutic delivery or microsurgery or combination thereof at a target location within or adjacent to the target volume.

FIGS. 1A-1B depict a non-limiting example of a treatment using a miniature device of the present disclosure. FIG. 1A depicts the untethered miniature device, delivered in the tumor using a biopsy-like tool, traveling in a tumor and creating a channel. Once the channel is completed, it is irrigated and filled with a therapeutic liquid, using the same biopsy-like tool. FIG. 1B depicts that in the hours post irrigation, the therapeutic liquid continues to diffuse deeper in the tumor.

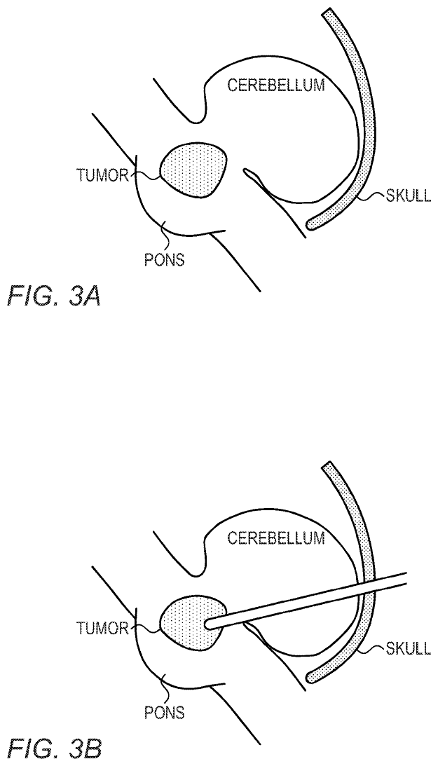

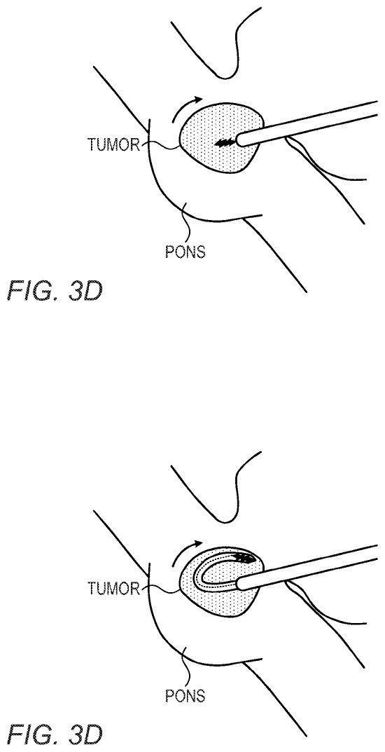

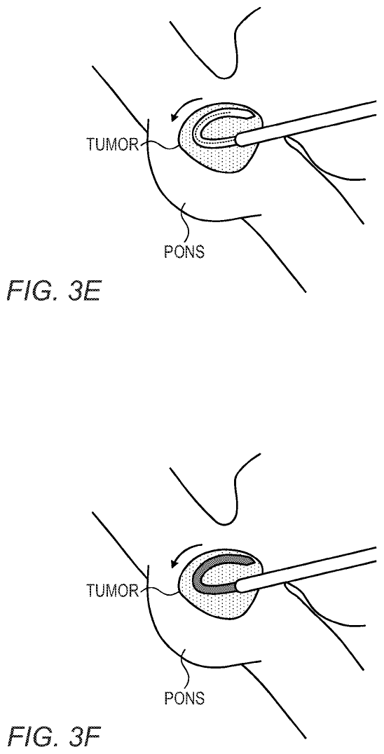

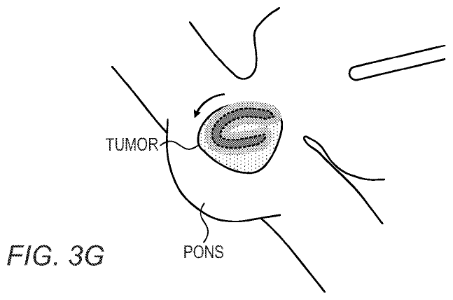

FIGS. 3A-3G also illustrate a non-limiting example of a method of using a system of the present disclosure. FIG. 3A depicts a tumor in the pons of a subject. The subject undergocs imaging (e.g., using MRI) and a plan for accessing the tumor is determined. FIG. 3B illustrates a needle accessing the tumor through a burr hole, e.g., using guidance. FIG. 3C illustrates a miniature device being introduced through the needle near or inside the tumor. FIG. 3D illustrates the miniature device travelling due to an external force along a curvilinear path (which can be a pre-determined path) and generating a channel inside or near the tumor. FIG. 3E depicts removal of the miniature device from the tissue. FIG. 3F depicts the filling of the channel created by the miniature device with a liquid therapeutic agent introduced through the needle. FIG. 3G depicts removal of the needle and diffusion of the agent into the tissue.

In some embodiments, the therapeutic or diagnostic agent may comprise any of a small-molecule drug or respective pro-drug, biologic, microbe (i.e., bacteria, archaea, and/or single-celled eukaryote), virus, micelles, nutrient, mineral, vitamin, peptide, enzyme, antibody or antibody fragment (whether engineered or natural), nucleic acid (including DNA and/or RNA), carbohydrate, lipid or fatty acid, aqueous solution, non-aqueous solution, nanomaterial, nanoparticle (e.g., immunogold nanoparticle), glue, binder, chemical suture, label (e.g., radiolabel), or any combination thereof.

In some embodiments, the agent delivered may be a label, such as, fluorescence labels, dycs, radiolabels, linked to e.g., antibodies, antibody constructs, antibody epitopes (including complete sequences, fragments, native epitopes, and engineered epitopes), ligands, hybridizable oligonucleotides (including, e.g., dsDNA, ssDNA, dsRNA, ssRNA, cDNA), cells and other labels known in the art.

In some embodiments, the therapeutic cargo comprises a cell, biologic, small molecule drug, sugar, lipid, fatty acid, vitamin, mineral, ion or salt, microbe, virus, nucleic acid, vector, peptide, or protein, or a combination thereof. These include, but are not limited to, peptoids, encoding DNA sequences, RNAi, siRNA, miRNA, shRNA, and AAV-based therapeutics.

In some embodiments, the therapeutic cargo comprises therapeutic radioisotope(s), including, but not limited to, ¹³¹I, ⁹⁹Tc, ¹⁰B, ⁶⁰Co. The radioisotope cargo can be delivered in any suitable configuration, e.g., as a solution, a specific formulation, or as beads (such as to provide brachytherapy).

The miniature device may function within the channel after having formed it, for example to affect the concentration of the therapeutic agent, to limit exposure of healthy tissue to the therapeutic agent, and/or for treatment of a diffuse condition which manifests at specific loci within the tissue. For example, as illustrated in FIG. 8A, the miniature device may remain at the end of the channel formed in the tissue. (In FIGS. 8A through 8E, the channel is indicated by a line representing the path it follows, and the stippled area indicates location of the therapeutic agent by representation of its diffusion within the tissue; while generally elliptical channels are shown, this is for illustration only, and in practice any suitably shaped channel may be formed). According to this example, after the therapeutic agent is administered into the channel, it is allowed to remain there, e.g., to diffuse within the tissue surrounding the channel. When it is determined that the therapeutic agent should be removed from the channel, the miniature device may be operated to traverse the channel in the reverse direction from that which it did to form the channel, thereby forcing excess therapeutic agent to be removed from within the channel.

As illustrated in FIG. 8B, the miniature device may form a channel comprising a closed channel portion, wherein the miniature device remains at the point where the two ends of the closed channel portion meet. According to this example, the miniature device may form most of the closed channel portion (for example, stopping at a position as shown in FIG. 8A), at which point the therapeutic agent is introduced, filling the channel. The miniature device is then operated to proceed until it meets an earlier point in the path thereby completing the closed channel portion. It remains there, isolating the closed channel portion from the rest of the channel, thereby facilitating retention of the therapeutic agent within the closed channel portion. A flushing liquid may be introduced into the channel, which removes the therapeutic agent which remains in the part of the channel downstream of the miniature device.

As illustrated in FIG. 8C, the miniature device may be configured to absorb therapeutic agent from within the channel. Accordingly, it may comprise an absorbent structure configured to absorb the therapeutic agent as it traverses the channel.

As illustrated in FIG. 8D, a second miniature device may be introduced to serve as an upstream stopper. According to this example, a first miniature device may remain at the end of the channel formed in the tissue. The therapeutic agent is introduced, after which a second miniature device is operated to travel within the channel, pushing the therapeutic agent ahead of it. The second miniature device stops at a predetermined location. The therapeutic agent remains in the portion of the channel between the first and second miniature devices. This may be useful, e.g., in ensuring that all the therapeutic agent which is introduced remains within the channel and a proper location therewithin, without requiring that any be flushed therefrom.

As illustrated in FIG. 8E, the method described above with reference to and illustrated in FIG. 8D may be modified to use a single channel to maintain quantities of therapeutic agent at separate points therealong. According to this example, a first miniature device remains at one end of the channel. A first quantity of therapeutic agent is introduced, and a second miniature device is operated to travel within the channel until it reaches a location at which the first two miniature devices define therebetween a location for maintaining a first quantity of therapeutic agent. A third miniature device is operated to travel within the channel until it reaches an upstream end of a second location for maintaining a quantity of the therapeutic agent. A second quantity of therapeutic agent is introduced, and a fourth miniature device is operated to travel within the channel until it reaches a location at which the third and fourth miniature devices define therebetween a location for maintaining the second quantity of therapeutic agent. This process may be repeated to maintain as many separate quantities of therapeutic agent as is necessary. It will be appreciated that as the quantities of therapeutic agent are isolated from each other, different types of therapeutic agents may be maintained at different areas along the channel.

Besides therapeutic and diagnostic agents, an activating agent that converts a prodrug into the therapeutic and/or the diagnostic agent may be administered via the channel, mutatis mutandis. In some embodiments of these embodiments, the prodrug is administered separately.

The activating agent may facilitate the conversion directly, i.e., by interacting with the prodrug, or indirectly, e.g., by facilitating production of an agent which directly interacts with the prodrug to convert it into the therapeutic agent. The activating agent may comprise an enzyme and/or an enzymatically active oligonucleotide.

The activating agent may encode an auxiliary activating agent, where the auxiliary activating agent is configured to directly interact with the prodrug to facilitate its conversion into the therapeutic agent. An extracellular, intracellular, and/or intranuclear process may express the auxiliary activating agent encoded by the activating agent. The activating agent may comprise an enzyme precursor, an oligonucleotide precursor, and/or a protease precursor. The activating agent may also comprise a vector configured for cellular delivery of the activating agent. The vector may be selected from an adeno-associated virus (AAV), a human immunodeficiency virus, a human papillomavirus sequence, one or more small molecules, one or more lipids, a peptide sequence, and a recognition sequence.

The activating agent may be selected from an endogenous or a non-endogenous human enzyme, a pro-enzyme, a construct encoding an active enzyme, and a cell/nuclear delivery sequence (e.g., a small molecule, a lipid, a specific receptor affinity sequence, an AAV-based delivery vector).

The activating agent may be, and/or may encode, a kinase, a phosphatase, a peptidase, a ligase, a lyase, a hydrolase, a protease, a deacetylase, a phosphodiesterase, an esterase, an amidase, a reductase, a phospholipase, or a cytochrome.

The prodrug may be administered by any suitable method, for example locally or systemically, e.g., orally, intravenously, intramuscularly, subcutaneously, intranasally, sub-buccally, intrathecally, intracerebroventrally, etc. The therapeutic agent is converted into a therapeutic agent at the target site by the activating agent, directly or indirectly, and remains inactive or partially inactive (e.g., dormant, inert, non-toxic, not bioavailable) until exposure to the activating agent and/or auxiliary activating agent produced thereby. Owing to the delivery of the activating agent only at the target site, only the portion of the prodrug which is in the vicinity of the activating agent is converted into the therapeutic agent, thereby providing localized treatment. The remainder of the prodrug, i.e., that which is not activated, is eliminated naturally by the patient.

It will be appreciated that administration of the prodrug does not have to be fully carried out in the order presented. For example, depending on the amount of time a therapeutic agent needs to reach a target site, step (administering of a prodrug) is performed in advance of generating the channel and filling it with the activating agent. According to other examples, generating the channel and filling it with the activating agent may be performed at a target site well in advance of treatment, with introduction of the prodrug performed at one or more points in the future.

Besides therapeutic agents, other therapeutic and/or diagnostic articles may be administered via the channel, mutatis mutandis. According to some examples, a conventional catheter or surgical tool may be inserted. A channel formed according to the present disclosure may allow placement of the catheter/tool along paths which would not otherwise be attainable according to conventional methods.

According to other examples, one or more electronic devices, e.g., neurostimulation and/or transmitting devices, may be deposited along the channel. Such devices may be placed temporarily or permanently, as required.

### External System

The external system is provided to control the miniature device within tissue to generate a channel in a targeted volume within a patient. According to some examples, it may comprise a software module and a hardware system. In a representative but not limiting example, the external system uses magnetic fields to exert mechanical forces on the miniature device and to control it to perform specific actions. The forces, controllably and predictably exerted on the miniature device, are expected to have various consequences on the milieu. In some embodiments, the external system mediates propulsion and navigation of the miniature device from one predetermined position to another. For example, the external system controls specific motion(s) of the miniature device including, but not limited to, standalone axial or diametral spinning, rotation, vibration, tumbling, crawling, rocking, lateral motion, etc.

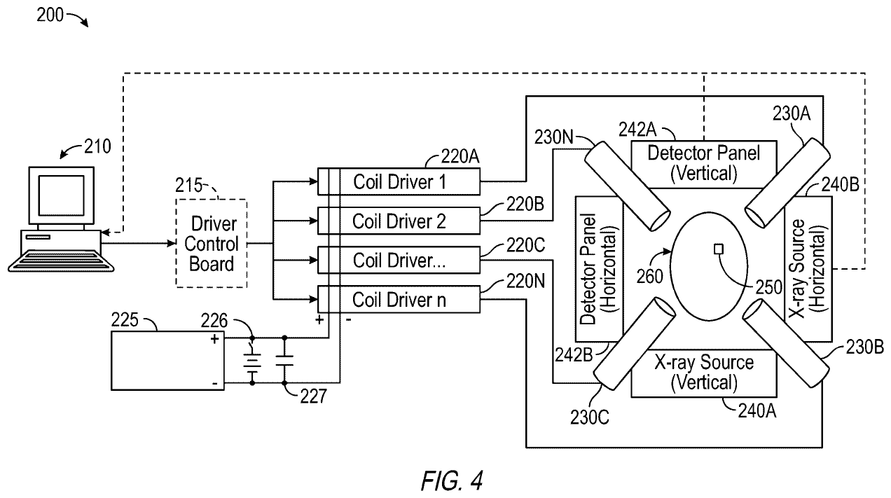

FIG. 4 schematically illustrates an external system 200 for providing a treatment to a target volume in accordance with embodiments the present disclosure. The external system 200 includes both hardware and software to achieve control of a miniature device within various milieu within the patient.

The external system uses magnetic fields to exert mechanical forces on the miniature device and to control it to perform specific actions. The forces, controllably and predictably exerted on the miniature device, are expected to have various consequences on the milieu. In some embodiments, the external system mediates propulsion and navigation of the miniature device from one predetermined position to another. For example, the external system controls specific motion(s) of the miniature device including, but not limited to, standalone axial or diametral spinning, rotation, vibration, tumbling, crawling, rocking or combined with lateral motion to drill or bore through tissue including, but not limited to, a brain tumor and adjacent tissue. The foregoing action could be performed once or multiple times. The foregoing action could be performed in a single or multiple loci to achieve the desired effect. For example, specific motion around a certain point accomplishes a desired primary effect. In another example, the forces coax the miniature device to remain immobile near a predetermined position.

In some embodiments, the external system generates magnetic fields using permanent magnets. In some embodiments, the permanent magnets can be mounted on a mechanical setup that holds the magnet and can move in three dimensions. In some embodiments, the permanent magnets can rotate. In some embodiments, there is only one permanent magnet. In other embodiments, there are several magnets that are actuated independently.

In some embodiments, the external system 200 can generate magnetic fields using electromagnets. An electromagnet comprises one, two, three or multiple coils. An electromagnet can, in addition, comprise a bobbin to support the coil and a yoke that modifies the electromagnetic properties of the coil. In some embodiments, the electromagnets are mounted on a mechanical setup that holds them at a fixed location with respect to the milieu where the miniature device resides. In some embodiments, the electromagnets are mounted on a mechanical setup that controllably and predictably determines the position of the electromagnets with respect to the milieu where the miniature device resides. In some embodiments, there is only one electromagnet. In some embodiments, there are electromagnets that are actuated according to predetermined control algorithms that take into the account the position, velocity, acceleration and pose of the miniature device extracted using separate algorithms that use X-ray images of the miniature device in real time. In some embodiments, the fields are generated in such a fashion that the forces applied to the miniature device cause it to rotate about its axis.

In some embodiments, the external system generates magnetic fields using a combination of electromagnets and permanent magnets. In some embodiments, both electromagnets and permanent magnets are used in concert to generate the fields and applies forces to the miniature device.

In some embodiments, the external system generates the magnetic fields using six to twelve electromagnetic coils. For example, eight electromagnetic coils are used to generate the magnetic fields. Each coil is between 4″×4″×4″ and 12″×12″×12″ in size, for example, each electromagnetic coil is 8″×8″×15″ in size. Each coil can carry up to 550 Amps, and for example runs at 30 Amps. In some embodiments, the electromagnetic coils have a ferromagnetic yoke. In other embodiments, the electromagnetic coils have no yoke. In some embodiments, the electromagnetic coils are arranged around the head of the subject in use. In some embodiments, the electromagnetic coils are between 5 to 25 cm, e.g., 15 cm, from the milieu.

The external system may use a variety of visualization or imaging systems to assist with the application of the forces and result in adequate control of the miniature device. In some embodiments, the external system uses X-rays to image the miniature device inside the targeted milieu. In some embodiments, the external system uses two X-rays applied in a stereovision system (e.g., biplane X-ray detectors) to image the miniature device inside the targeted milieu to determine its three-dimensional position with respect to landmarks or fiducial markers used as a reference. In some embodiments, the external system uses optical stereovision to determine the position of the electromagnets or permanent magnets with respect to fiducial markers or landmarks used as a reference. In some embodiments, the external system uses a combination of X-ray stereovision and optical stereovision to monitor the position of the miniature device, only visible under X-ray vision, with respect to the external electromagnets or permanent magnets, only visible under optical vision. In another embodiment, the external system includes radiopaque markers on the electromagnets or permanent magnets coils and includes these markers within the field-of-view of the X-ray system to allow the control system to determine the relative position of the coils in reference to the miniature device. The control system can utilize the relative position between the coils and the miniature device in controlling the magnetic gradients generated to propel the miniature device.

The external system can include one or more software modules including a planning software submodule and a hardware-control software submodule. In some embodiments, the external system software modules can use pre-recorded digital data, such as magnetic resonance imaging (MRI) scans, computed tomography (CT) scans, etc., to assist with the activities performed by both submodules. In some embodiments, MRI scans are analyzed by the software module and digital three-dimensional objects representing various tissue masses present in the host environment are generated automatically. In some embodiments, the hardware-control software module uses digital information from the optical or X-ray stereovision system and computes automatically the mathematical transformation to allow display, in a single referential, three-dimensional data generated from MRI, vision and X-ray system.

In this example, the external system 200 can include components such as computing system 210, driver control board 215, magnetic coil drivers 220A-220N (collectively referenced as coil drivers 220), magnetic coils 230A-230N (collectively referenced as coils 230), X-ray sources 240A, 240B, and X-ray detectors 242A, 242B. In an example, the driver control board 215 can be integrated into the computing system 210. Power is provided to the external system 200 by the power supply 225 that can also include an optional battery bank 226 and an optional capacitor bank 227. The power supply 225 converts standard 110/220 volt electrical sources into high and low voltage direct current (DC) as needed within the external system 200. The battery bank 226 and/or capacitor bank 227 can be utilized to assist in supplying power during short duration peak power demands.

Computing system 210 can perform the various control procedures and processes described herein. In general, pre-operative treatment plans can be accessed by the computing system 210 through a network or other storage device. The computing system 210 can utilize the pre-operative plans to control (e.g., navigation and propel) a miniature device 250 from an introduction site to a target volume within the patient 260 through selective activation of the coils 230 via the driver control board 215 sending command signals to the coil drivers 220. The coils 230 can be configured to generate different levels of magnetic gradients at various frequencies as needed to produce the desired propulsion of the miniature device 250 (discussed in greater detail below).

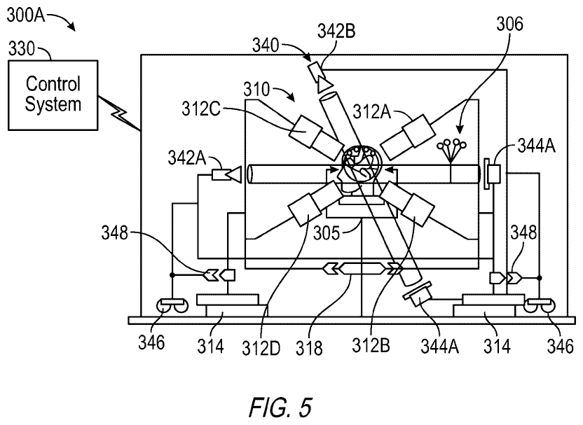

FIG. 5 schematically illustrates an external system 300A for delivering a treatment to a target volume using magnetic propulsion in accordance with embodiments of the present disclosure. In this example, the external system 300A is illustrated as including components such as: a biplane X-ray system 340 with table and head-holder 305; an electromagnetic array 310 capable of generating design magnetic field gradients; a control system 330; a miniature device; and tools (e.g., a biopsy needle or similar needle) to introduce and retrieve the miniature. In this example, the external system 300A is designed for treatment of a localized area of anatomy, such as the brain. The electromagnetic array 310 includes a plurality of coils, such as coil 312A, coil 312B, coil 312C, and coil 312D (collectively referenced as coils 312). The coils 312 are designed to generate magnetic fields to propel and control a miniature device within the target area of anatomy. In this example, the electromagnetic array 310 is affixed to a rail system 314 that allows for precise movement of the electromagnet array 310 along a linear path. The rail system 314 can dock to the table and head-holder 305 via docking mechanism 318. In order to navigate a miniature device within the target anatomy, the external system 300A system includes a stereo X-ray system, biplane X-ray system 340. The biplane X-ray system 340 includes two pairs of emitters and receivers offset from each other at a known angle. The biplane system 340 includes emitter 342A paired with receiver 344A as well as emitter 342B paired with receiver 344B. The biplane X-ray system 340 is also affixed to a rail system, rail system 346. The biplane X-ray system 340 also optionally includes docking mechanism 348 to dock to the electromagnet array 310. An additional part of a navigation system can optionally include a navigation array 306 that is affixed directly to the head holder 305. The external system 300A includes a control system 330 that communicates with both the electromagnetic array 310 and the biplane X-ray system 340 to control operation of the external system 300A.

A potential clinical implementation of the methods and systems described herein could involve development of a treatment plan, potentially using MRI scans made prior to the procedure. The MRI scan with treatment plan information is subsequently merged with a live X-ray view. The system detects the miniature device and provides a magnetic field that propels the miniature device as defined in the treatment plan. As a safety feature, fluoroscopy frequency is potentially adjusted based on the speed of the miniature device.

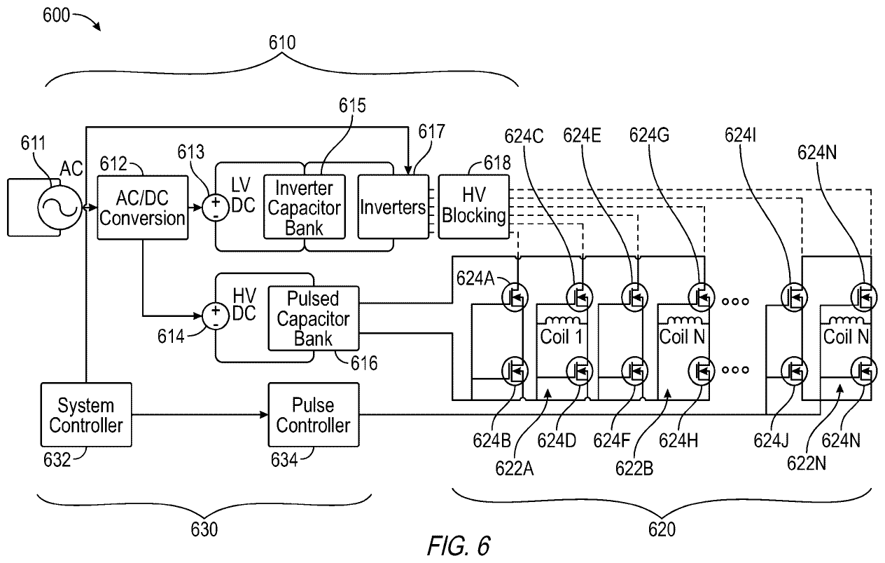

FIG. 6 is a block diagram illustrating an external system 600 for providing a treatment to a target volume. More specifically, the external system 600 is a magnetic propulsion and control system that uses two power circuits to run a single array of magnetic coils 620 including a plurality of coils 622A-622N (collectively referenced as magnetic coils 620). In this example, one power circuit is designed to have high accuracy and low power, enabling movement of a miniature device in anatomical fluids by current drivers. The second power circuit in this example is a low accuracy high-power circuit that enables movement through tissue by controlled discharge of a large capacitor bank, such as pulsed capacitor bank 616.

The external system 600 outlined in the block diagram can utilize a method involving connecting a plurality of electromagnets to a plurality of solid-state relays to produce a configurable arrangement of circuits that can be energized by an energy storage system. In this example, the electromagnets are placed in symmetric positions around an isocenter and receive a sequence of electrical pulses from the energy storage system to produce a magnet field with arbitrary polarization at the isocenter. The magnetic field is received by the miniature device that translates the magnetic energy into a force in a single direction with each electrical pulse. In this example, the position of the miniature device can be measured by an external position measurement unit (such as through real-time medical imaging and analysis of the images). The amplitude of the electrical pulse is controllable, which translates into a controllable pulsating force on the miniature device. In an example, the amplitude of the pulse is controlled by the solid-state relays. In another example, the amplitude of the pulse is controlled by the charge state of the energy storage system. The solid-state relays can be operatively connected to a controller (such as control system 630) such that the controller can alter the configuration of the circuits connected to the electromagnets. Control of the solid-state relays can be done in response to position of the miniature device, with an algorithm updating commands delivered to the solid-state relays in response to position of the miniature device.

Essentially, the control system 630 is connected to a multi-leg bidirectional converter with independent high and low-side FETs/IGBTs, which can be used to pulse all of the magnetic coils 620 with any polarity, and optionally using PWM. This allows the external system to command synchronized pulses in arbitrary directions at a high rate.

In the example illustrated in FIG. 6, the external system 600 can include three primary components a power system 610, an array of magnetic coils 620, and a control system 630. The power system 610 can include an alternating current (AC) power supply 611 coupled to an AC/DC converter 612. The AC/DC converter 612 converts the incoming AC power into direct current (DC) power used to energize the magnetic coils 620. In this example, the AC/DC converter 612 feeds a low voltage DC supply 613 (LVDC supply 613) and a high voltage DC (HVDC supply 614) supply 614. The LVDC supply 613 charges an inverter capacitor bank 615 which is connected to the DC input of the Inverters 617. The inverters 617 are electrically connected to the Coils 622A-622N through an HV Blocking device 618, which isolates the inverters 617 output from the pulsed capacitor bank 616, which is charged by the HVDC supply 614. The solid-state relays 624A-624N are connected to the pulse controller 634, which switches the state of the relays in order to deliver power with the correct polarity and to the correct coils. The pulse controller 634 and the inverters 617 are connected to the system controller 632, which ensures that each power system is operating appropriately per the commands of the system controller 632.

### Miniature Device

The miniature device may be provided as described in PCT application PCT/US2022/40303. The miniature device's dimensions, geometry, etc., are selected to facilitate performance of its mission within the milieu. FIGS. 2A-2D depict non-limiting examples of three miniature devices of the present disclosure. Each of the miniature devices comprise a material that responds to an external magnetic field. FIG. 2A depicts a miniature device that is shaped like a corkscrew. FIG. 2B is a cross section through the corkscrew shaped miniature device in the presence of a rotating magnetic field (B). The miniature device is induced to rotate by the rotating magnetic field and is set in motion due to its corkscrew like geometry. FIG. 2C depicts a miniature device that resembles a wood screw. The wood screw shaped miniature device responds to rotating magnetic fields and is set in motion due to its fins or threads. FIG. 2D depicts a miniature device that resembles a bullet, which responds to field gradients.

In some embodiments, the miniature device is elongated in one dimension. In some embodiments, the miniature device has a total length between about 1 mm and about 20 mm, for example about 7 mm. In some embodiments, the miniature device has an outer diameter between about 1 mm and about 5 mm, for example about 2.5 mm. In some embodiments, the miniature device has a total length between about 50 μm and about 10,000 μm. In some embodiments, the miniature device has an outer diameter between about 50 μm and about 50,000 μm.

In some embodiments, the body is made of or comprises a permanent magnet magnetized along a longitudinal axis of the miniature device. In some embodiments, the permanent magnet is magnetized along a direction that is substantially perpendicular to a longitudinal axis of the miniature device. In some embodiments, the body is made of a radiopaque material. In some embodiments, the body is made of a rare earth magnet, coated with electroplated nickel and gold, ceramic, plastic, or other substantially non-magnetic biocompatible material.

In some embodiments, the body of the miniature device is made from a neodymium magnet. In some embodiments, the grade of the neodymium magnet is from N40 to N55, e.g., N50. In some embodiments, the magnet's residual induction (Br) is between about 12 kg and about 15 kg, e.g., about 14 kg. In some embodiments, (BH) max is between about 38 MGOe and about 56 MGOe, e.g., about 47 MGOe. In some embodiments, the magnet's intrinsic coercivity, (Hci) is greater than about 11 kOe. In some embodiments, the magnet's intrinsic normal coercivity, (HcB) is greater than about 10 kOe. In some embodiments, the magnet's maximum operating temperature is about 60° C. to about 80° C., preferably about 80° C.

In some embodiments, the miniature device is configured to be driven by a static unidirectional force, for example arising from the interaction between the magnetic field generated by the external system and a permanent magnet of the miniature device.

The system may serve as an introduction tool, i.e., be provided to introduce the miniature device to a site near or within the targeted volume. The tools of the present disclosure include biopsy needles or other needles that have similar profiles and attributes. The tool may be designed with specific features to minimize tissue damage, along the path to the region of interest. The tool has a lumen that extends from its proximal end to its distal end. In use, the tool is placed through a burr hole and allows fluid or devices, such as the miniature devices described herein, to travel from the proximal end of the tool that resides outside the body to the distal end near or within the targeted volume.

In some embodiments, the introduction tool comprises a sharp rigid pointed surgical instrument fitted with a needle or cannula with an inner diameter that can accommodate the miniature device. In some embodiments, the cannula is rigid and made of titanium or other non-magnetic metal or plastic. In some embodiments, the cannula is flexible. In some embodiments, the cannula outer diameter ranges from about 1 mm to about 10 mm, for example about 3.5 mm. In some embodiments, the cannula is stabilized by a mechanical arm. In some embodiments, the cannula is designed to be stabilized by a user. In some embodiments, the cannula is configured to be guided using X-rays. In some embodiments, the cannula is configured to be guided using a stereotactic technique.

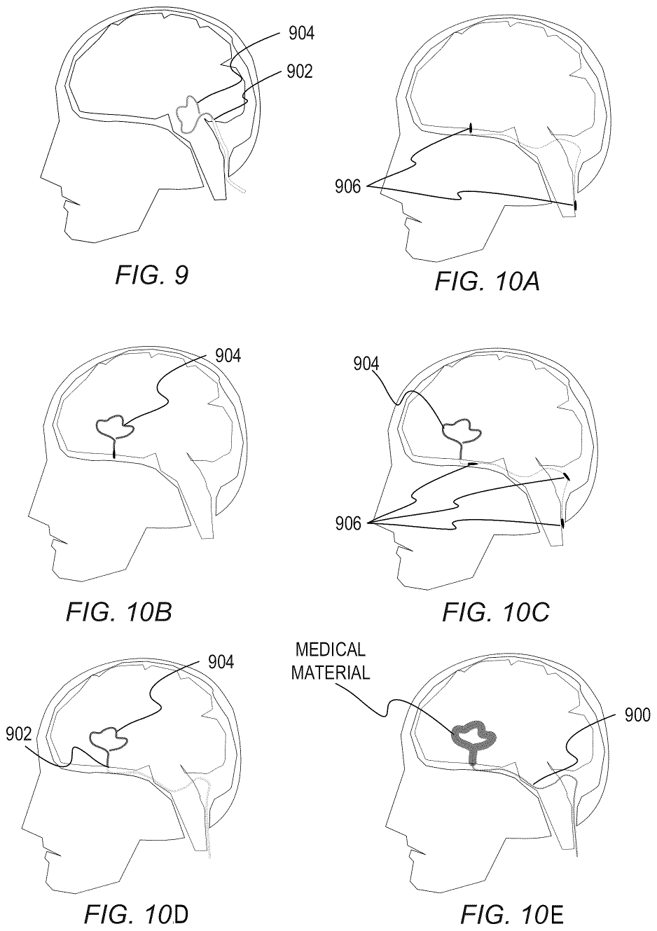

For example, as illustrated in FIG. 9, a hollow introduction tool 900, comprising an opening 902 at its distal end, is inserted into the patient such that its opening is located at or near the targeted volume. A miniature device is introduced into the targeted volume via the introduction tool 900, and is operated to form a channel 904 in the target volume beyond the distal end of the introduction tool. According to some examples, the miniature device is navigated back to the introduction tool 900 for retrieval, and subsequently medical material, such as a therapeutic or diagnostic agent, a prodrug, a device, etc., is delivered to the channel 904 via the introduction tool 900. According to some examples, medical material is delivered to the channel 904 via the introduction tool 900 prior to retrieval of the miniature device, for example as described above.

According to some examples, for example as illustrated in FIGS. 10A through 10E, the miniature device 906 is navigated to the target volume (FIG. 10A; e.g., if the target volume is in the brain, the miniature device may be navigated through cerebrospinal fluid). The miniature device is operated to form a channel 904 in the target volume (FIG. 10B), and subsequently is navigated away from the target volume, for example exiting the patient (FIG. 10C). An introduction tool 900 is steered within the patient to the channel 904, such that its distally located opening 902 is adjacent the channel 904, for example lying in registration therewith (FIG. 10D). According to some examples, a miniature device may be used to assist the introduction tool 900 to reach the target volume. Medical material is then delivered to the channel 904 via the introduction tool 900 (FIG. 10E).

In some embodiments, the tool is also used to retrieve the miniature device from the patient, for example once the mission is complete.

In some embodiments, the external system monitors the position of the tool using a stereo vision camera. In some embodiments, the route of the tool is planned in advance by medical personnel as described herein. In some embodiments, the route is charted using MRI data. In some embodiments, the position of the tool is represented, as perceived by an optical stereovision system, in real time, on an electronic display station.

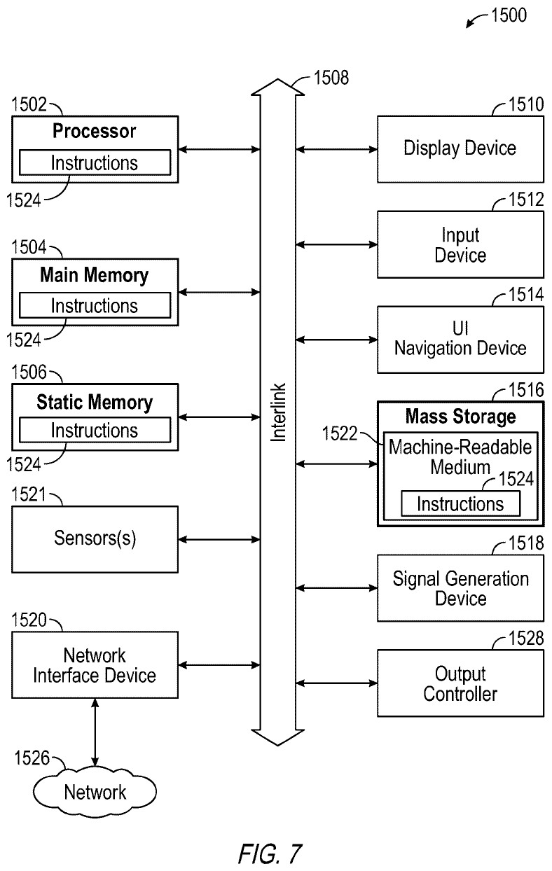

FIG. 7 illustrates a block diagram of an example machine 1500 upon which any one or more of the procedures (processes) discussed herein may be performed in accordance with some embodiments. The example machine 1500 can also be used to operate the control system 330 discussed above in reference to FIG. 5 as well as the control system 630 discussed above in reference to FIG. 6. In alternative embodiments, the machine 1500 may operate as a standalone device and/or may be connected (e.g., networked) to other machines. In a networked deployment, machine 1500 may operate in the capacity of a server machine, a client machine, or both in server-client network environments. In an example, machine 1500 may act as a peer machine in peer-to-peer or other distributed network environment. Machine 1500 may be a personal computer, a tablet computer, a set-top box, a personal digital assistant, a mobile telephone, a web appliance, a network router, switch or bridge, or any other suitable machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 1500 may include a hardware processor 1502 (e.g., a central processing unit, a graphics processing unit, a hardware processor core, or any combination thereof), a main memory 1504 and a static memory 1506, some or all of which may communicate with each other via an interlink (e.g., bus) 1508. Machine 1500 may further include a display unit 1510, an alphanumeric input device 1512 (e.g., a keyboard), and a user interface navigation device 1514 (e.g., a mouse). In an example, the display unit 1510, input device 1512 and user interface navigation device 1514 may be a touch screen display. Machine 1500 may additionally include a storage device (e.g., drive unit) 1516, a signal generation device 1518 (e.g., a speaker), a network interface device 1520, and one or more sensors 1521, such as a global positioning system sensor, compass, accelerometer, or another sensor. The machine 1500 may include an output controller 1528, such as a serial (e.g., Universal Serial Bus, parallel, or other wired or wireless (e.g., infrared, near field communication, etc.) connection to communicate and/or control one or more peripheral devices (e.g., a printer, card reader, etc.).

The storage device 1516 may include a machine readable medium 1522 on which is stored one or more sets of data structures or instructions 1524 (e.g., software) embodying or utilized by any one or more of the techniques or functions described herein. The instructions 1524 may also reside, completely or at least partially, within the main memory 1504, within static memory 1506, or within the hardware processor 1502 during execution thereof by the machine 1500. In an example, one or any combination of the hardware processor 1502, the main memory 1504, the static memory 1506, or the storage device 1516 may constitute machine readable media.

While the machine readable medium 1522 is illustrated as a single medium, the term “machine readable medium” may include a single medium or multiple media (e.g., a centralized or distributed database, and/or associated caches and servers) configured to store the one or more instructions 1524. The term “machine readable medium” may include any medium that is capable of storing, encoding, or carrying instructions for execution by the machine 1500 and that cause the machine 1500 to perform any one or more of the techniques of the present disclosure, or that is capable of storing, encoding, or carrying data structures used by or associated with such instructions. Non-limiting machine-readable medium examples may include solid-state memories, and optical and magnetic media.

The instructions 1524 may further be transmitted or received over a communications network 1526 using a transmission medium via the network interface device 1520 utilizing any one of a number of transfer protocols (e.g., frame relay, internet protocol, transmission control protocol, user datagram protocol, hypertext transfer protocol, etc.). Example communication networks may include a local area network, a wide area network, a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer networks, among others. In an example, the network interface device 1520 may include one or more physical jacks (e.g., Ethernet, coaxial, or phone jacks) or one or more antennas to connect to the communications network 1526. In an example, the network interface device 1520 may include a plurality of antennas to wirelessly communicate using at least one of single-input multiple-output, multiple-input multiple-output, or multiple-input single-output techniques. The term “transmission medium” shall be taken to include any intangible medium that is capable of storing, encoding, or carrying instructions for execution by the machine 1500, and includes digital or analog communications signals or other intangible medium to facilitate communication of such software.

Method/procedure examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories, read only memories, and the like.

It will be appreciated that while the system and method described herein relate to forming a channel using untethered miniature devices controlled by external non-contact, e.g., magnetic, forces, this is by way of example only. According to some aspects, the channel may be formed by any suitable means, including, but not limited to, untethered miniature devices, tethered miniature devices, conventional devices (e.g., using a needle- and/or catheter-based approach), etc. Moreover, the therapeutic/diagnostic activity performed within the channel may be carried out using any suitable device, e.g., a treatment miniature device, and is not limited to the methods described herein.

One or more components of the system may be provided, mutatis mutandis, as described in any one or more of WO 2019/213368, WO 2019/213362, WO 2019/213389, WO 2020/014420, WO 2020/092781, WO 2020/092750, WO 2018/204687, WO 2018/222339, WO 2018/222340, WO 2019/212594, WO 2019/213368, WO 2019/005293, WO 2020/096855, WO 2020/252033, WO 2021/021800, WO 2021/092076, WO 2021/126905, WO 2021/216463, WO 2022/119816, WO 2022/246170, and WO 2022/246174, the full contents of which are incorporated herein by reference.

The various embodiments described herein can be combined to provide further embodiments. All U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for delivering a medical material to a target volume of solid tissue in a patient, the method comprising:
forming an intraparenchymal channel along a predefined path, at least a portion of the predefined path being within or adjacent to the target volume; and
delivering the medical material via the channel to the target volume.

2. The method according to claim 1, wherein the delivery of medical material comprises administering a fluid or gel comprising a therapeutic and/or diagnostic agent into the channel.

3. The method according to claim 2, wherein the therapeutic and/or diagnostic agent is selected from a small molecule, a peptide, a peptoid, an oligonucleotide sequence, a nucleic acid, an oncolytic virus, an endogenous cell, and/or an engineered cell.

4. The method according to claim 1, wherein the delivery of medical material comprises administering a fluid or gel comprising an activating agent that converts a prodrug into a therapeutic and/or diagnostic agent into the channel.

5. The method according to any one of claims 2 through 4, wherein the administering comprises pumping the fluid or gel into the channel.

6. The method according to any one of the preceding claims, wherein the delivery of medical material comprises inserting one or more surgical tools through the channel to perform therapeutic delivery or microsurgery at a target location within or adjacent to the target volume.

7. The method according to any one of the preceding claims, wherein the delivery of medical material comprises installing one or more neurostimulation and/or neurodiagnostic devices in the channel at the target location.

8. The method according to any one of the preceding claims, wherein the delivery of medical material comprises navigating an untethered treatment miniature device to the target location via the channel.

9. The method according to any one of the preceding claims, further comprising:
providing a system comprising:
a miniature device configured to be maneuvered by magnetic fields; and
an external system configured to generate the magnetic fields to propel and navigate the miniature device in the target volume of tissue; and
operating the external system to propel and navigate the miniature device to travel along the predefined path to form the channel.

10. The method according to claim 9, wherein the same or an additional miniature device controls local exposure to the therapeutic agent to spare healthy tissue.

11. The method according to any one of claims 9 and 10, further comprising retrieving the miniature device from the patient before delivery of medical material.

12. The method according to claim 11, wherein retrieving the miniature device includes navigating and propelling the miniature device into a needle.

13. The method according to any one of claims 9 through 12, wherein the miniature device has a corkscrew shape.

14. The method according to any one of claims 9 through 12, wherein the miniature device has a wood screw shape.

15. The method according to any one of claims 9 through 14, further comprising imaging the miniature device in real time within the target volume.

16. The method according to claim 1, further comprising:
receiving output from a biplane X-ray system communicatively coupled to a control system of a magnetic propulsion system that propels the miniature device; and
determining, within the control system from the output, a position and orientation of the miniature device within the patient.

17. The method according to claim 16, wherein the determining comprises determining a relative location of an array of magnetic coils in reference to the miniature device from the output from the biplane X-ray system; and adjusting propulsion parameters based on the relative location.

18. The method according to any one of claims 9 through 17, the system further comprising a needle having a proximal end and a distal end and a lumen extending from the distal end to the proximal end, the method further comprising:
introducing the needle through a burr hole in the patient's skull, such that the proximal end resides outside the patient's body and the distal end is placed near or within the targeted volume brain tissue; and
inserting the miniature device into the proximal end of the needle and releasing the miniature device from the distal end of the needle into a starting location of the predetermined path.

19. The method according to claim 18, wherein the needle is introduced via a trans-peduncular approach.

20. The method according to any one of the preceding claims, wherein the solid volume is in brain tissue.

21. The method according to any one of the preceding claims, wherein the target volume comprises tumor tissue.

22. The method according to claim 21, wherein the tumor tissue is a brainstem glioma.

23. The method according to claim 22, wherein the brainstem glioma is diffuse intrinsic pontine glioma.

24. The method according to claim 21, wherein the tumor tissue is in the pons of the patient.

25. The method according to any one of the preceding claims, wherein the predetermined path comprises a curvilinear portion.

26. The method according to claim 25, wherein the curvilinear portion of the predetermined path is within the target volume.

27. The method according to any one of claims 25 and 26, wherein the curvilinear portion is three-dimensional.

28. The method according to any one of claims 4 through 27, further comprising the step of administering the prodrug to the patient.

29. The method according to claim 28, wherein the activating agent comprises an enzyme and/or an enzymatically active oligonucleotide.

30. The method according to claim 28, wherein said activating agent encodes an auxiliary activating agent, and said auxiliary activating agent converts the prodrug into the therapeutic agent by directly interacting with the prodrug.

31. The method according to claim 30, wherein an extracellular, intracellular, and/or intranuclear process expresses the auxiliary activating agent encoded by the activating agent.

32. The method according to claim 32, wherein the activating agent comprises an enzyme precursor, an oligonucleotide precursor, and/or a protease precursor.

33. The method according to claim 32, wherein said activating agent further comprises a vector configured for cellular delivery of the activating agent.

34. The method according to claim 33, wherein the vector is selected from a group including an adeno-associated virus, a human immunodeficiency virus, a human papillomavirus sequence, one or more small molecules, one or more lipids, a peptide sequence, and a recognition sequence.

35. The method according to claim 28, wherein the activating agent is and/or encodes one or more enzymes selected from a kinase, a phosphatase, a peptidase, a ligase, a lyase, a hydrolase, a protease, a deacetylase, a phosphodiesterase, an esterase, an amidase, a reductase, a phospholipase, or a cytochrome.

36. The method according to any one of claims 28 through 35, wherein the prodrug is administered before the activating agent.

37. The method according to according to any one of claims 28 through 36, wherein the prodrug is administered systematically.

38. A system configured for performing the method according to any one of the preceding claims.
